(19) United States
(12) Patent Application Publication
(10) Pub. No.: US 2013/0095040 A1
(43) Pub. Date: Apr. 18, 2013

(54) DYE COMPOSITIONS AND DYE SYNTHESES

(71) Applicant: Umamaheshwar P. Mokkapati, Bangalore (IN)

(72) Inventor: Umamaheshwar P. Mokkapati, Bangalore (IN)

(73) Assignee: GE HEALTHCARE AS, Oslo (NO)

(21) Appl. No.: 13/805,055

(22) PCT Filed: Jun. 29, 2011
(86) PCT No.: PCT/EP2011/060936

§ 371 Date: Dec. 18, 2012

(30) Foreign Application Priority Data
Jun. 29, 2010 (GB) 1010878.5
Jun. 30, 2010 (GB) 1010993.2

(51) Int. Cl. C09B 23/08; C09B 23/01; C09B 57/00; A61K 49/00

(52) U.S. Cl. CPC C09B 23/083; A61K 49/001; C09B 23/0091; C09B 57/00
USPC 424/9.1; 548/455; 548/507; 548/509; 548/494; 548/502

(57) ABSTRACT

The present invention relates to sulfonated unsymmetrical pentamethine optical dye compositions, especially dyes suitable for biological applications in vitro, and for in vivo imaging. Improved dye compositions and intermediates are provided, which enable the suppression of undesirable newly-identified impurities. Also provided is the use of the improved dye compositions in the preparation of conjugates with biological targeting molecules.

### FIELD OF THE INVENTION

The present invention relates to the field of sulfonated unsymmetrical pentamethine optical dyes, especially dyes suitable for biological applications in vitro, and for in vivo imaging. Improved dye compositions and intermediates are provided, which enable the suppression of undesirable newly-identified impurities.

### BACKGROUND TO THE INVENTION

The sulfonation of dyes by the introduction of sulfonate (—SO₃H or —SO₃) substituents is an established method of increasing the water solubility of the dye.

WO 2005/044923 and US 2007/0203343 A1 disclose the synthesis of new class of sulfonated pentamethine and heptamethine cyanine dyes useful for the labelling and detection of biological molecules. Included is the synthesis of the unsymmetrical cyanine dye Cy7, and its N-hydroxysuccinimide (NHS) ester:

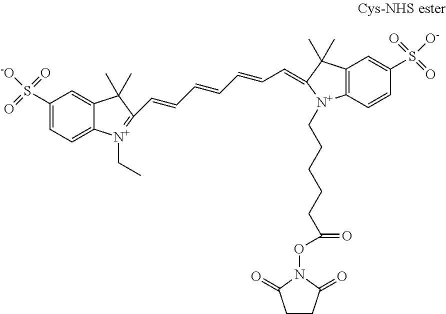

WO 2005/123768 discloses conjugates of sulfonated cyanine dyes with RGD peptides, and the use of the conjugates in diagnostic optical imaging techniques.

Jiang et at [Tet. Lett., 48 5825-5829 (2007)] disclose an efficient approach to the synthesis of unsymmetrical water-soluble cyanine dyes using poly(ethylene glycol) as a soluble support.

WO 2008/139206 discloses the synthesis of a specific class of unsymmetrical pentamethine cyanine dyes and their use as imaging agents for in vivo optical imaging. Example 3 of WO 2008/139206 provides a synthesis of the dye Cy5**:

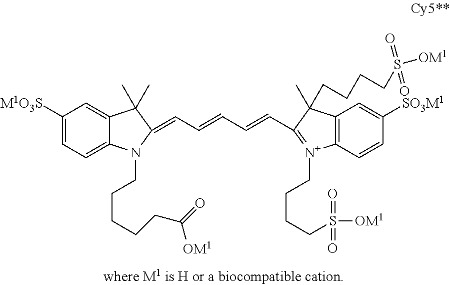

Prior art dye syntheses, especially of unsymmetrical cyanine dyes, do however suffer from low yields with complex, labour-intensive purification methods (eg. preparative HPLC), which are most suited to milligramme scale due to sample loading considerations. When used for biological applications, especially in vivo optical imaging, there is a need for such dyes being obtainable in multi-gramme quantities, at pharmaceutical grade, with suppression of unwanted impurities.

### THE PRESENT INVENTION

The present invention provides compositions useful in the synthesis of unsymmetrical sulfonated optical dyes, wherein previously unrecognised impurities are identified and suppressed. Identification and control of such impurities is important for Good Manufacturing Practice (GMP). Dye intermediate compositions useful in the synthesis of unsymmetrical optical dyes are also provided, as well as dye preparation methods which provide improved dye compositions by suppression of key impurities.

The present invention permits the preparation of sulfonated unsymmetrical pentamethine cyanine dyes in gramme quantities, at pharmaceutical grade, with suppression of unwanted impurities. The compositions and methods are particularly useful when preparing such sulfonated dyes for biological applications, especially when preparing conjugates of the dyes with biological molecules for in vivo optical imaging.

### DETAILED DESCRIPTION OF THE INVENTION

In a first aspect, the present invention provides a dye composition which comprises an unsymmetrical cyanine dye of Formula A, together with symmetrical cyanine dyes of Formula B and Formula C:

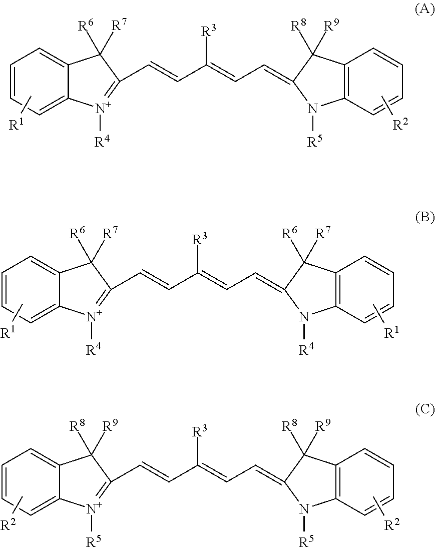

characterised in that said dye composition comprises at least 92% of A, together with less than 2% of B and less than 2% of C,
wherein within said composition:
   R¹ and R² are independently H, —SO₃M¹ or R^{a}, where M¹ is H or B^{c}, and B^{c} is a biocompatible cation;
   R³ is H or an R^{d} group;
   R⁴ and R⁵ are independently an R^{d} group;
   R⁶ to R⁹ are independently C₁₋₃ alkyl or an R^{c} group, and are chosen such that in Formula A, at least one of R⁶ to R⁹ is R^{c};
   R^{a} is C₁₋₄ sulfoalkyl;
   R^{b} is C₁₋₆ carboxyalkyl;
   R^{c} is an R^{a} or R^{b} group;
   R^{d} is C₁₋₅ alkyl or an R^{c} group;
with the provisos that in Formula A:
(i) at least one of the pairs of substituents R¹/R², R⁴/R⁵, R⁶/R⁸ and R⁷/R⁹ is different;
(ii) the cyanine dye comprises at least one R^{b} group and a total of 3 to 6 sulfonic acid substituents from the R¹, R², R^{a} and R^{c} groups.

The term “composition” means a mixture of the compounds of Formulae A, B and C plus possibly other components. The composition can be in dry form, or can be a mixture in solution.

By the term “symmetrical” is meant that, in eg. Formula B, within the Formula each pair of R¹, R⁴, R⁶ and R⁷ groups is the same, so that the pattern of substituents on each indole head group moiety at the termini of the methine chain is the same. By the term “unsymmetrical” is meant that, in Formula A, at least one of R¹, R⁴, R⁶ and R⁷ is different from their counterpart substituents on the other head group R², R⁵, R⁸ and R⁹ respectively. The result is that the pattern of substituents on each indole head group moiety at the termini of the methine chain is different.

By the term “biocompatible cation” (B^{c}) is meant a positively charged counterion which forms a salt with an ionised, negatively charged group (in this case a sulfonate group), where said positively charged counterion is also non-toxic and hence suitable for administration to the mammalian body, especially the human body. Examples of suitable biocompatible cations include: the alkali metals sodium or potassium; the alkaline earth metals calcium and magnesium; and the ammonium ion. Preferred biocompatible cations are sodium and potassium, most preferably sodium.

By the term “comprises less than 2% of the symmetrical cyanine dye” is meant that the composition comprises less than 2.0 mole percent of the symmetrical cyanine dye of Formula B, and less than 2.0 mole percent of the symmetrical cyanine dye Formula C.

The “dyes” which can be prepared using the compositions of the invention include cyanine dyes. The dyes are sulfonated. By the term “sulfonated” is meant that said dyes have at least one sulfonic acid substituent. By the term “sulfonic acid substituent” is meant a substituent of formula —SO₃M¹, where M¹ is H or B^{c}, and B^{c} is a biocompatible cation. The —SO₃M¹, substituent is covalently bonded to a carbon atom, and the carbon atom may be aryl or alkyl.

Whilst some unsymmetrical cyanine dyes of Formula A were known in the prior art, the unsymmetrical cyanine dye compositions of the first aspect were not understood, because the identities of the key impurities were not known. In addition to identifying such impurities, the present invention provides methods for controlling such impurities to give the improved compositions. The symmetrical impurity dyes of Formula B and C, having similar optical properties, might interfere with the biological applications of dyes of Formula A in vitro and in vivo. Once formed and present in the composition, these impurity dyes of have similar characteristics to the desired unsymmetrical dye. Consequently they are difficult to separate chromatographically on a preparative scale, and hence suppression of their formation in the first place is key. The present invention solves that problem by providing dye intermediates and synthetic processes whereby the impurities are suppressed from the outset and at key stages throughout the synthesis. If the impurity symmetric dyes are not suppressed in the dye composition, they may also form conjugates when the dye is conjugated to a biological targeting moiety, or synthetic polymer—resulting in reduced yield and increasing problems in purification of the conjugate.

The term “comprising” has its conventional meaning throughout this application and implies that the composition must have the components listed, but that other, unspecified compounds or species may be present in addition. The term ‘comprising’ includes as a preferred subset “consisting essentially of” which means that the composition has the components listed without other compounds or species being present.

### Preferred Aspects.

The dye composition of the first aspect preferably comprises at least 94% of A, together with less than 1.5% of B and less than 1.5% of C.

In Formula B, each R⁴ group is preferably the same R^{a} group. In Formula C, each R⁵ is preferably the same R^{b} group.

In the dye composition, R³ is preferably H. R⁶ to R⁹ are preferably chosen such that one (only) of R⁶ to R⁹ is an R^{c} group, and the other 3 are each C₁₋₂ alkyl, preferably CH₃. The single R^{c} group of R⁶ to R⁹ is preferably an R^{a} group.

The dye composition of the first aspect preferably comprises less than 1%, more preferably less than 0.5%, most preferably less than 0.3% of the acylated indole of Formula H, as defined in the fifth embodiment (below).

The dye composition of the first aspect preferably further comprises less than 1% of sulfonate ester derivatives of Formula A¹, B¹ or C¹, which correspond to the compounds of Formula A, B or C respectively wherein at least one of R¹ and R² is —SO₂—OR^{d}, where R^{d} is as defined above. More preferably, the dye composition comprises less than 0.5% of such sulfonate ester derivatives. Most preferably less than or equal to 0.3%. The present inventors have found that prior art syntheses provided Compound 5 having 2-2.5% of such sulfonate ester impurities. Such impurities are problematic, since they have the potential to react further when the dye is conjugated to eg. a biological targeting moiety or synthetic polymer.

The —SO₂—OR^{d} substituent is termed a “sulfonate ester” because the definitions of R^{d} require that an —SO₂—OC— ester covalent bond is present. Such sulfonate esters are previously unknown impurities in dye compositions of the first aspect. They arise from undesirable O-alkylation, in addition to the desired N-alkylation in the synthesis of dye intermediates. The suppression of said sulfonate esters is important, since they represent likely fluorescent, more lipophilic impurities in the desired sulfonated cyanine dye. Thus, a negatively charged, fully-ionised sulfonic acid substituent has been changed to a non-ionic, sulfonate ester.

The unsymmetrical cyanine dye of Formula A preferably has a total of 4 sulfonic acid substituents chosen from the R¹, R², R^{a} and R^{c} groups. The R^{a} groups are preferably of formula —(CH₂)ₖSO₃M′, where M′ is H or B^{c}, k is an integer of value 1 to 4, and B′ is a biocompatible cation (as defined above). k is preferably 3 or 4.

In the dye composition of the first aspect, R¹ and R² are preferably the same, more preferably both are SO₃M′. When R¹ and R² are both SO₃M′, the SO₃M′ substituents are preferably in the 5-position of the indole/indoline rings.

The dye of Formula A comprises at least one, more preferably one R^{b} group, i.e. a carboxyalkyl substituent. That makes the dye bifunctional, by providing a functional group (carboxyl) through which the dye can be attached to a biological targeting moiety (BTM), as is described in the ninth aspect (below). It is preferred to have one such carboxyalkyl substituent, so that the dye has a sole site of attachment to the BTM.

An especially preferred unsymmetrical cyanine dye is of Formula IA:

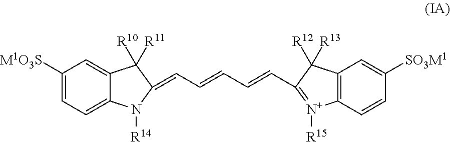

where:
R¹⁴ and R¹⁵ are independently R^{c} groups;

R¹⁰ to R¹³ are independently C₁₋₅ alkyl or R^{c} groups, and are chosen such that either R¹⁰═R¹¹═R^{e} or R¹²═R¹³═R^{e}, where R^{e} is C₁₋₂ alkyl;

R^{c} and M¹ are as defined above for Formula A.

The R^{a} groups of Formula IA are preferably independently —(CH₂)ₖSO₃M¹, where k is an integer of value 1 to 4, and k is preferably 3 or 4. Preferably the dyes of Formula IA have one C₁₋₆ carboxyalkyl (i.e. R^{b}) substituent to permit facile covalent attachment to biological molecules.

Preferred dyes of Formula IA are chosen such that one of R¹⁰ to R¹³ is an R^{c} group, and the others are each R^{e} groups, most preferably with each R^{c} equal to CH₃. Especially preferred dyes of Formula IA are of Formula IB, wherein one of R¹⁰ to R¹³ is an R^{a} group, and the others are each R^{e} groups, most preferably each equal to CH₃. Preferred dyes of Formula IA have one of the R^{c} groups chosen to be C₁₋₆ carboxyalkyl.

Most preferred specific dyes of Formulae IA and IB respectively are Alexa Fluor™ 647 and Cy5**, with Cy5** being the ideal:

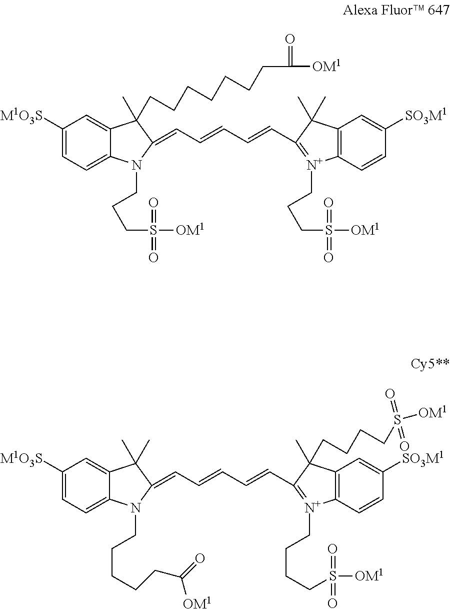

The dye compositions of the first aspect can be obtained as described in the seventh aspect (below).

The purities of the dye composition are quoted via analytical HPLC determination (area under the curve) at four different wavelengths—214 nm, 273 nm, 450 nm and 650 nm. The identities of the components of the composition were determined by LCMS and confirmed by NMR.

In a second aspect, the present invention provides a dye intermediate composition which comprises a hemicyanine dye of Formula E, and an amidine salt of Formula Z:

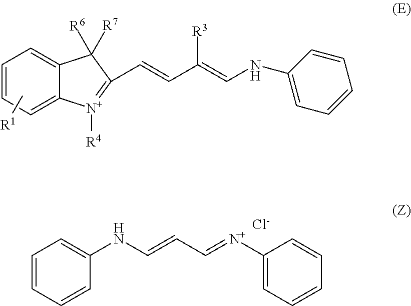

characterised in that said dye composition comprises at least 92% of E, together with less than 2% of Z,
wherein, within said composition, R¹, R³, R⁴, R⁶ and R⁷ and preferred embodiments thereof are as defined in the first aspect.

The dye intermediate compositions of the second aspect can be obtained as described in the fourth aspect (below). The amidine salt of Formula Z is commercially available from eg. Sigma-Aldrich.

The dye intermediate composition of the second aspect has an important contribution to achieving the desired unsymmetrical dye composition of the first aspect. See the fourth aspect (below). The dye intermediate composition preferably comprises less than 0.5% of Z, more preferably less than 0.3% of Z.

In a third aspect, the present invention provides a dye head group composition which comprises an indolinium salt of Formula F, and an indole of Formula G, characterised in that said composition comprises at least 92% of F, and less than 3% of G:

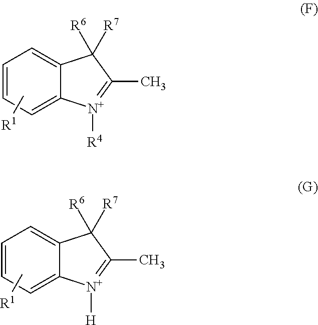

wherein, within said composition:
   R¹ and R⁴ and preferred aspects thereof are as defined in the first aspect;
   R⁶ and R⁷ are independently C₁₋₃ alkyl or an R^{c} group, and are chosen such that in Formulae F and G, at least one of R⁶ and R⁷ is an R^{c} group,
   where R^{c} and preferred aspects thereof is as defined in the first aspect.

The term “head group” means the group at one or other terminus of the polymethine group of an optical dye, suitably a cyanine dye.

For the head group composition of the third aspect, R^{c} is preferably an R^{a} group, and preferred aspects thereof, as defined in the first aspect. It is preferred that one of R⁶ and R⁷ is an R^{a} group, and the other is an R^{e} group—where R^{a} and R^{e} and preferred aspects thereof are as described in the first aspect (above).

The dye head group composition also preferably contains less than 1% of the acylated indole of Formula H as defined in the fifth aspect (below). For Compound 1, the acylated indole of Formula H is a significant potential impurity and hence, if not controlled or removed, it can be carried over into subsequent steps of the unsymmetrical dye synthesis (see Scheme 1).

The dye head group compositions of the third aspect can be obtained as described in the sixth aspect (below).

In a fourth aspect, the present invention provides a method of preparation of the dye intermediate composition of the second aspect, which comprises reaction of the dye head group composition of the third aspect with 1.05 to 1.25 molar equivalents of the amidine salt of Formula Z as defined in the second aspect.

The reaction of the sixth aspect is carried out in a suitable solvent, preferably chosen from: sulfolane, N,N-dimethylacetamide or 1,4-butane sultone.

Thus, the present inventors have found that, if present in the dye intermediate composition, the indolinium salt of Formula F reacts in the next step to give the symmetrical impurity dye of Formula B. If an excess of the amidine salt Z is present in the dye intermediate composition, it will react with the indolinium salt of Formula J (below) to form the undesirable symmetrical impurity dye of Formula C.

In order to suppress the formation of such symmetrical dyes, a slight excess of amidine salt Z is used to completely consume the indolinium salt of the dye head group composition, followed by a purification to remove excess of Z in the dye intermediate composition. and thereafter adding exactly one mole equivalent of the indolinium salt of Formula J to suppress the formation of symmetrical dyes.

Purification of Compound 3 of the present invention is achieved by dissolving it in methanol at 55° C. and precipitating it with 2-propanol and filtering the precipitated solid. The amidine salt does not precipitate from the solvent mixture.

In a fifth aspect, the present invention provides an indole composition which comprises the indole of Formula G as defined in the third aspect, and the acylated indole of Formula H, characterised in that said composition comprises at least 90% of G, and less than 5% of H:

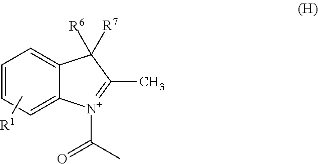

wherein:
   R¹ is as defined in the first aspect;
R⁶ and R⁷ are independently are independently C₁₋₃ alkyl or an R^{c} group, and are chosen such that in Formulae G and H, at least one of R⁶ and R⁷ is an R^{c} group.

Preferred aspects of the indole of Formula G in the fifth aspect are as defined in the third aspect (above). Preferred aspects of R¹, R⁶ and R⁷ in the fifth aspect are as defined in the first aspect (above). The indole composition preferably comprises less than 2% of H, more preferably less than 1% of H.

The present inventors have found that prior art syntheses of indole compositions of the fifth aspect comprise 10-15% of Compound H. That impurity would be carried over unchanged in the next step of the unsymmetrical dye synthesis (see Scheme 1), and hence it is important to suppress it at the earliest opportunity.

For the conversion of Compound 1 to Compound 2, the temperature and quantity of the solvent have been found to be the key parameters studied to prevent formation of N-acetyl indole of Formula H as an impurity in the reaction. A product with greater than 90% HPLC purity was obtained when the temperature of the reaction was reduced to 100° C. and the quantity of solvent was 25-30 times (by weight) with respect to 4-hydrazinobenzenesulfonic acid (4-HBSA). Using such techniques, the present invention suppressed Compound H in Compound 2 to less than 1%.

In a sixth aspect, the present invention provides a method of preparation of the head group composition of the third aspect, which comprises reaction of the indole composition of the fifth aspect with an alkylating agent of formula R⁴—X, where R⁴ is as defined in the first aspect, and X is a leaving group.

The term “alkylating agent” in the sixth aspect has its conventional meaning in organic chemistry. The term “leaving group” has its conventional meaning in organic chemistry in this regard. Suitable such leaving groups (X) include: X=Hal, where Hal is halogen, or sulfonate ester (such as mesylate, tosylate or triflate). For the introduction of sulfoalkyl substituents, the use of cyclic sulfonate esters, i.e. sultones, eg. 1,4-butane sultone, as the alkylating agent is preferred. For R⁴—X, and X=Hal, Hal is preferably Cl, Br or I, most preferably Br. Suitable such alkylating agents are commercially available.

The reaction of the sixth aspect is carried out in a suitable solvent. The suitable solvent can be: sulfolane, N,N-dimethylacetamide and sultone (sultone can be used as both solvent and reagent). The reaction is suitably carried out at 100-150° C., preferably about 140° C. for about 48-hours.

The present inventors have found that such indole alkylation reactions typically lead to N-alkylated indoles of moderate purity (65-70%), and having the free indole (N—H) starting material content at a level of 7 to 8%. The indole composition of the fifth aspect is prepared by suspending the crude indolinium salt of Formula F, having the indole of Formula G present at about 7-8% in methanol and heating it to reflux, together with a catalytic amount (ca. 0.15 molar equivalents) of triethylamine to facilitate dissolution—via formation of a salt which is soluble in methanol. Subsequent precipitation of the desired product was achieved with by adding 0.2 to 0.5%, preferably 0.3% aqueous 2-propanol. Use of higher water content (2-3%) renders the product sticky thereby making the filtration process impossible. For Compound 2, when a catalytic quantity of triethylamine was used for purification, the Compound 1 impurity level was diminished by ca. 85-90% thereby increasing the purity of Compound 1 to 92-95%. The diminution of impurity Compound 1 was only 10-15% when purification was done without the use of triethylamine.

In a seventh aspect, the present invention provides a method of preparation of the dye composition of the first aspect, which comprises reaction of the dye intermediate composition of the second aspect, with one molar equivalent an indolinium salt of Formula J:

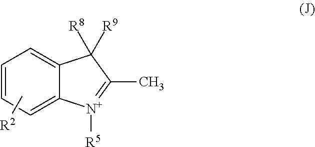

wherein:
   R² and R⁵ are as defined in the first aspect;
   R⁸ and R⁹ are independently C₁₋₃ alkyl or an R^{c} group, where R^{c} is as defined in the first aspect.

Preferred aspects of the dye intermediate composition in the seventh aspect, are as described in the second aspect. Preferred aspects of R², R⁵, R⁸ and R⁹ in the seventh aspect are as described in the first aspect.

As described in the fourth aspect (above), it is important to use exactly one molar equivalent of the indolinium salt of Formula J (based on the hemicyanine dye of Formula E). Otherwise, the risk of symmetrical dye impurities being generated is increased.

In an eighth aspect, the present invention provides a pharmaceutical composition which comprises the dye composition of the first aspect, in a biocompatible carrier medium, in sterile form suitable for mammalian administration.

The “biocompatible carrier medium” comprises one or more pharmaceutically acceptable adjuvants, excipients or diluents. It is preferably a fluid, especially a liquid, in which the compound of Formula (I) is suspended or dissolved, such that the composition is physiologically tolerable, i.e. can be administered to the mammalian body without toxicity or undue discomfort. The biocompatible carrier medium is suitably an injectable carrier liquid such as sterile, pyrogen-free water for injection; an aqueous solution such as saline (which may advantageously be balanced so that the final product for injection is either isotonic or not hypotonic); an aqueous solution of one or more tonicity-adjusting substances (eg. salts of plasma cations with biocompatible counterions), sugars (e.g. glucose or sucrose), sugar alcohols (eg. sorbitol or mannitol), glycols (eg. glycerol), or other non-ionic polyol materials (eg. polyethyleneglycols, propylene glycols and the like). The biocompatible carrier medium may also comprise biocompatible organic solvents such as ethanol. Such organic solvents are useful to solubilise more lipophilic compounds or formulations. Preferably the biocompatible carrier medium is pyrogen-free water for injection, isotonic saline or an aqueous ethanol solution. The pH of the biocompatible carrier medium for intravenous injection is suitably in the range 4.0 to 10.5.

The pharmaceutical composition may optionally contain additional excipients such as an antimicrobial preservative, pH-adjusting agent, filler, stabiliser or osmolality adjusting agent. By the term “antimicrobial preservative” is meant an agent which inhibits the growth of potentially harmful micro-organisms such as bacteria, yeasts or moulds. The antimicrobial preservative may also exhibit some bactericidal properties, depending on the dosage employed. The main role of the antimicrobial preservative(s) of the present invention is to inhibit the growth of any such micro-organism in the pharmaceutical composition. The antimicrobial preservative may, however, also optionally be used to inhibit the growth of potentially harmful micro-organisms in one or more components of kits used to prepare said composition prior to administration. Suitable antimicrobial preservative(s) include: the parabens, i.e. methyl, ethyl, propyl or butyl paraben or mixtures thereof; benzyl alcohol; phenol; cresol; cetrimide and thiomersal. Preferred antimicrobial preservative(s) are the parabens.

The term “pH-adjusting agent” means a compound or mixture of compounds useful to ensure that the pH of the composition is within acceptable limits (approximately pH 4.0 to 10.5) for human or mammalian administration. Suitable such pH-adjusting agents include pharmaceutically acceptable buffers, such as tricine, phosphate or TRIS [ie. tris(hydroxymethyl)aminomethane], and pharmaceutically acceptable bases such as sodium carbonate, sodium bicarbonate or mixtures thereof. When the composition is employed in kit form, the pH adjusting agent may optionally be provided in a separate vial or container, so that the user of the kit can adjust the pH as part of a multi-step procedure.

By the term “filler” is meant a pharmaceutically acceptable bulking agent which may facilitate material handling during production and lyophilisation. Suitable fillers include inorganic salts such as sodium chloride, and water soluble sugars or sugar alcohols such as sucrose, maltose, mannitol or trehalose.

The pharmaceutical compositions may be prepared under aseptic manufacture (ie. clean room) conditions to give the desired sterile, non-pyrogenic product. It is preferred that the key components, especially the associated reagents plus those parts of the apparatus which come into contact with the imaging agent (eg. vials) are sterile. The components and reagents can be sterilised by methods known in the art, including: sterile filtration, terminal sterilisation using e.g. gamma-irradiation, autoclaving, dry heat or chemical treatment (e.g. with ethylene oxide). It is preferred to sterilise some components in advance, so that the minimum number of manipulations needs to be carried out. As a precaution, however, it is preferred to include at least a sterile filtration step as the final step in the preparation of the pharmaceutical composition.

Preferred aspects of the dye in the pharmaceutical composition in the eighth aspect, are as described in the first aspect.

In a ninth aspect, the present invention provides the use of the dye composition of the first aspect, or the pharmaceutical composition of the eighth aspect, in the preparation of a conjugate of the unsymmetrical dye of Formula A with a biological targeting moiety or a synthetic macromolecule.

The use of the ninth aspect includes a method of preparation of said conjugate starting from the dye composition of the first aspect, or the pharmaceutical composition of the eighth aspect. The dye-BTM conjugates of this aspect have applications both in vitro and in vivo.

By the term “biological targeting moiety” (BTM) is meant a compound which, after administration, is taken up selectively or localises at a particular site of the mammalian body in vivo. Such sites may for example be implicated in a particular disease state or be indicative of how an organ or metabolic process is functioning.

By the term “synthetic macromolecule” is meant a polymer of molecular weight 2 to 100 kDa, preferably 3 to 50 kDa, most preferably 4 to 30 kDa. The polymer can be a polyamino acid such as polylysine or polyglycollic acid, or a polyethyleneglycol (PEG). The term ‘synthetic’ is as defined below.

The BTM may be of synthetic or natural origin, but is preferably synthetic. The term “synthetic” has its conventional meaning, i.e. man-made as opposed to being isolated from natural sources eg. from the mammalian body. Such compounds have the advantage that their manufacture and impurity profile can be fully controlled. Monoclonal antibodies and fragments thereof of natural origin are therefore outside the scope of the term ‘synthetic’ as used herein.

The molecular weight of the BTM is preferably up to 30,000 Daltons. More preferably, the molecular weight is in the range 200 to 20,000 Daltons, most preferably 300 to 18,000 Daltons, with 400 to 16,000 Daltons being especially preferred. When the BTM is a non-peptide, the molecular weight of the BTM is preferably up to 3,000 Daltons, more preferably 200 to 2,500 Daltons, most preferably 300 to 2,000 Daltons, with 400 to 1,500 Daltons being especially preferred.

The biological targeting moiety preferably comprises: a 3-100 mer peptide, peptide analogue, peptoid or peptide mimetic which may be a linear or cyclic peptide or combination thereof; a single amino acid; an enzyme substrate, enzyme antagonist enzyme agonist (including partial agonist) or enzyme inhibitor; receptor-binding compound (including a receptor substrate, antagonist, agonist or substrate); oligonucleotides, or oligo-DNA or oligo-RNA fragments.

By the term “peptide” is meant a compound comprising two or more amino acids, as defined below, linked by a peptide bond (ie. an amide bond linking the amine of one amino acid to the carboxyl of another). The term “peptide mimetic” or “mimetic” refers to biologically active compounds that mimic the biological activity of a peptide or a protein but are no longer peptidic in chemical nature, that is, they no longer contain any peptide bonds (that is, amide bonds between amino acids). Here, the term peptide mimetic is used in a broader sense to include molecules that are no longer completely peptidic in nature, such as pseudo-peptides, semi-peptides and peptoids. The term “peptide analogue” refers to peptides comprising one or more amino acid analogues, as described below. See also “Synthesis of Peptides and Peptidomimetics”, M. Goodman et al, Houben-Weyl E22c, Thieme.

By the term “amino acid” is meant an L- or D-amino acid, amino acid analogue (eg. naphthylalanine) or amino acid mimetic which may be naturally occurring or of purely synthetic origin, and may be optically pure, i.e. a single enantiomer and hence chiral, or a mixture of enantiomers. Conventional 3-letter or single letter abbreviations for amino acids are used herein. Preferably the amino acids of the present invention are optically pure. By the term “amino acid mimetic” is meant synthetic analogues of naturally occurring amino acids which are isosteres, i.e. have been designed to mimic the steric and electronic structure of the natural compound. Such isosteres are well known to those skilled in the art and include but are not limited to depsipeptides, retro-inverso peptides, thioamides, cycloalkanes or 1,5-disubstituted tetrazoles [see M. Goodman, Biopolymers, 24, 137, (1985)]. Radio labelled amino acids such as tyrosine, histidine or proline are known to be useful in vivo imaging agents.

When the BTM is an enzyme substrate, enzyme antagonist, enzyme agonist, enzyme inhibitor or receptor-binding compound it is preferably a non-peptide, and more preferably is synthetic. By the term “non-peptide” is meant a compound which does not comprise any peptide bonds, ie. an amide bond between two amino acid residues. Suitable enzyme substrates, antagonists, agonists or inhibitors include glucose and glucose analogues such as fluorodeoxyglucose; fatty acids, or elastase, Angiotensin II or metalloproteinase inhibitors. A preferred non-peptide Angiotensin II antagonist is Losartan. Suitable synthetic receptor-binding compounds include estradiol, estrogen, progestin, progesterone and other steroid hormones; ligands for the dopamine D-1 or D-2 receptor, or dopamine transporter such as tropanes; and ligands for the serotonin receptor.

The BTM is most preferably a 3-100 mer peptide or peptide analogue. When the BTM is a peptide, it is preferably a 4-30 mer peptide, and most preferably a 5 to 28-mer peptide.

When the BTM is an enzyme substrate, enzyme antagonist, enzyme agonist or enzyme inhibitor, preferred such biological targeting molecules of the present invention are synthetic, drug-like small molecules i.e. pharmaceutical molecules. Preferred dopamine transporter ligands such as tropanes; fatty acids; dopamine D-2 receptor ligands; benzamides; amphetamines; benzylguanidines, iomazenil, benzofuran (IBF) or hippuric acid.

When the BTM is a peptide, preferred such peptides include:
somatostatin, octreotide and analogues,
peptides which bind to the ST receptor, where ST refers to the heat-stable toxin produced by E. coli and other micro-organisms;
bombesin;
vasoactive intestinal peptide;
neurotensin;
laminin fragments eg. YIGSR, PDSGR, IKVAV, LRE and KCQAGTFALRGDPQG,
N-formyl chemotactic peptides for targeting sites of leucocyte accumulation,
Platelet factor 4 (PF4) and fragments thereof,
RGD (Arg-Gly-Asp)-containing peptides, which may eg. target angiogenesis [R. Pasqualini et al., Nat. Biotechnol. 1997 June; 15(6):542-6]; [E. Ruoslahti, Kidney Int. 1997 May; 51(5):1413-7].
peptide fragments of α₂-antiplasmin, fibronectin or beta-casein, fibrinogen or thrombospondin. The amino acid sequences of α₂-antiplasmin, fibronectin, beta-casein, fibrinogen and thrombospondin can be found in the following references: α₂-antiplasmin precursor [M. Tone et al., J. Biochem, 102, 1033, (1987)]; beta-casein [L. Hansson et al, Gene, 139, 193, (1994)]; fibronectin [A. Gutman et al, FEBS Lett., 207, 145, (1996)]; thrombospondin-1 precursor [V. Dixit et al, Proc. Natl. Acad. Sci., USA, 83, 5449, (1986)]; R. F. Doolittle, Ann. Rev. Biochem., 53, 195, (1984);
peptides which are substrates or inhibitors of angiotensin, such as: angiotensin II Asp-Arg-Val-Tyr-Ile-His-Pro-Phe (E. C. Jorgensen et al, J. Med. Chem., 1979, Vol 22, 9, 1038-1044) [Sar, Ile] Angiotensin II: Sar-Arg-Val-Tyr-Ile-His-Pro-Ile (R. K. Turker et al., Science, 1972, 177, 1203).
Angiotensin I: Asp-Arg-Val-Tyr-Ile-His-Pro-Phe-His-Leu.

When the BTM is a peptide, one or both termini of the peptide, preferably both, have conjugated thereto a metabolism inhibiting group (M^{IG}). Having both peptide termini protected in this way is important for in vivo imaging applications, since otherwise rapid metabolism would be expected with consequent loss of selective binding affinity for the BTM peptide. By the term “metabolism inhibiting group” (M^{IG}) is meant a biocompatible group which inhibits or suppresses enzyme, especially peptidase such as carboxypeptidase, metabolism of the BTM peptide at either the amino terminus or carboxy terminus. Such groups are particularly important for in vivo applications, and are well known to those skilled in the art and are suitably chosen from, for the peptide amine terminus:

N-acylated groups —NH(C═O)R^{G} where the acyl group —(C═O)R^{G} has R^{G} chosen from: C₁₋₆ alkyl, C₃₋₁₀ aryl groups or comprises a polyethyleneglycol (PEG) building block. Preferred such amino terminus M^{IG} groups are acetyl, benzyloxycarbonyl or trifluoroacetyl, most preferably acetyl.

Suitable metabolism inhibiting groups for the peptide carboxyl terminus include: carboxamide, tert-butyl ester, benzyl ester, cyclohexyl ester, amino alcohol or a polyethyleneglycol (PEG) building block. A suitable M^{IG} group for the carboxy terminal amino acid residue of the BTM peptide is where the terminal amine of the amino acid residue is N-alkylated with a C₁₋₄ alkyl group, preferably a methyl group. Preferred such M^{IG} groups are carboxamide or PEG, most preferred such groups are carboxamide.

General methods for conjugation of cyanine dyes to biological molecules are described by Licha et at [Topics Curr. Chem., 222, 1-29 (2002); Adv. Drug Deliv. Rev., 57, 1087-1108 (2005)]. Peptide, protein and oligonucleotide substrates for use in the invention may be labelled at a terminal position, or alternatively at one or more internal positions. For reviews and examples of protein labelling using fluorescent dye labelling reagents, see “Non-Radioactive Labelling, a Practical Introduction”, Garman, A. J. Academic Press,1997; “Bioconjugation—Protein Coupling Techniques for the Biomedical Sciences”, Aslam, M. and Dent, A., Macmillan Reference Ltd, (1998). Protocols are available to obtain site specific labelling in a synthesised peptide, for example, see Hermanson, G. T., “Bioconjugate Techniques”, Academic Press (1996).

The invention is illustrated by the following, non-limiting Examples. The Compounds referred to are shown in FIG. 1, and the reaction summary in Scheme 1. Example 1 provides the synthesis of Compound 1, a specific sulfonated indole used in the synthesis of Compound 2. Compound 2 is a specific dye of Formula F of the dye head group composition of the third aspect. Example 2 provides the preparation of Compound 2 via N-alkylation of Compound 1, and its purification to provide a specific dye head group composition of the dye head group composition of the third aspect. Example 3 provides the synthesis of Compound 3, a specific hemicyanine dye of Formula E of the dye intermediate composition of the second aspect. Example 4 provides the synthesis of Compound 4, an indolinium salt of Formula J having a carboxyalkyl substituent. Example 5 provides the synthesis of Compound 5 (Cy5**), a specific dye of Formula A of the dye composition of the first aspect. Example 6 provides the HPLC purification and analysis of a dye composition comprising Compound 5.

The synthesis of the present invention is summarised in Scheme 1 for Compound 5 (Cy5**). The reaction is carried out in four steps, first preparing the indole composition of the fifth embodiment and Compound ! (Formula G). Secondly, preparing Compound 2 via N-alkylation of Compound 1 with 1,4-butane sultone. In the third step, the hemicyanine dye intermediate composition of the second aspect comprising Compound 3 is prepared via reaction of the head group composition with Compound Z as shown. The intermediate composition is preferably isolated and/or purified before use in the next step. In the final step, the intermediate composition is converted to the desired unsymmetrical dye composition of the first aspect by reaction with a specific indolinium salt of Formula J (Compound 4).

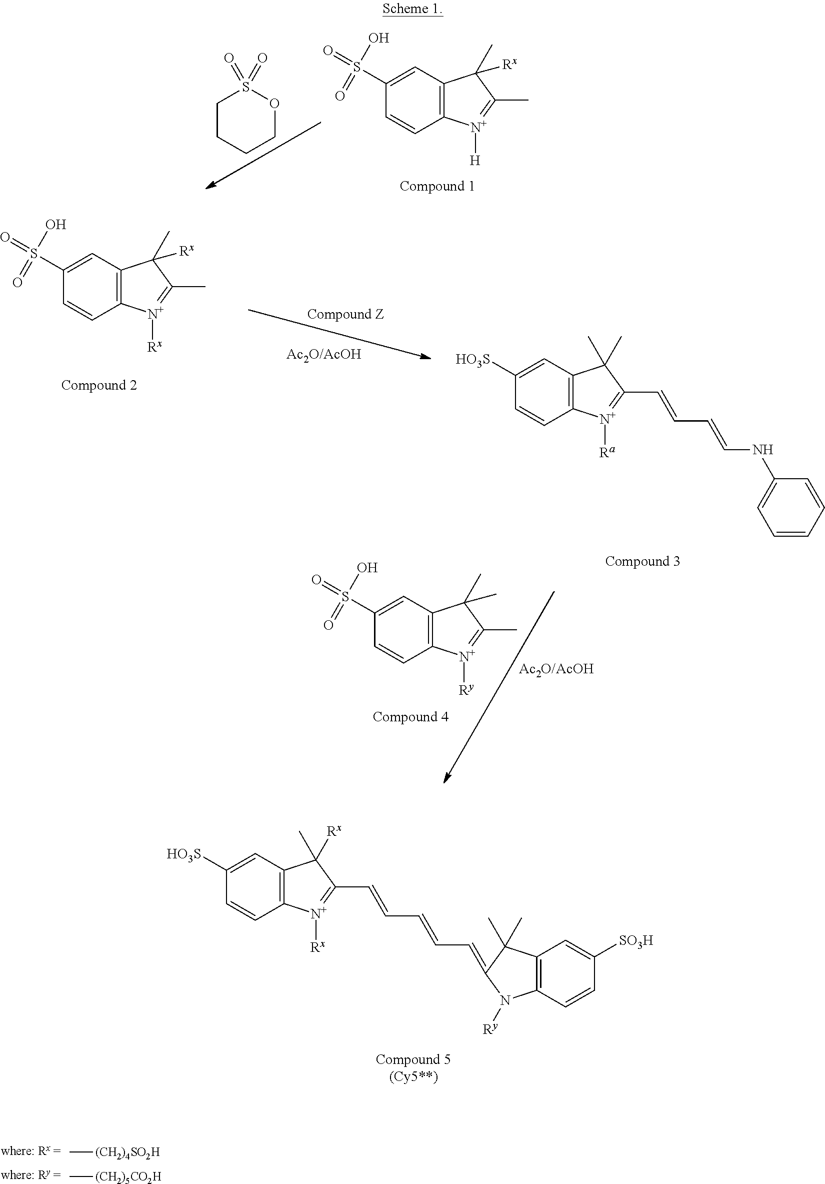

FIG. 1: Specific Compounds of the Invention.

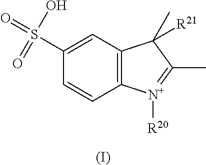

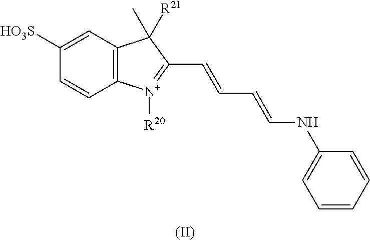

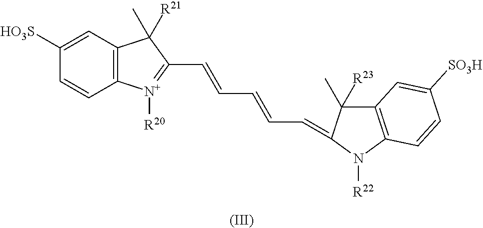

| Cmpd | Formula | R²⁰ | R²¹ | R²² | R²³ |
|---|---|---|---|---|---|
| 1 | I | —H | —(CH₂)₄SO₃H | — | — |
| 2 | I | —(CH₂)₄SO₃H | —(CH₂)₄SO₃H | — | — |
| 3 | II | —(CH₂)₄SO₃H | —(CH₂)₄SO₃H | — | — |
| 4 | I | —(CH₂)₅CO₂H | —CH₃ | — | — |
| 5 | III | —(CH₂)₄SO₃H | —(CH₂)₄SO₃H | —(CH₂)₅CO₂H | —CH₃ |
| Cy5** | | | | | |
| 6 | III | —(CH₂)₄SO₃H | —(CH₂)₄SO₃H | —(CH₂)₄SO₃H | —(CH₂)₄SO₃H |
| 7 | III | —(CH₂)₅CO₂H | —CH₃ | —(CH₂)₅CO₂H | —CH₃ |

### EXPERIMENTAL

### Example 1

### Synthesis of Disodium 2,3-dimethyl-3-(4-sulfonatobutyl)-3H-indole-5-sulfonate (Compound 1)

### Step (a)

### Sodium 5-(ethoxycarbonyl)-5-methyl-6-oxoheptane-1-sulfonate

Sodium hydride (29.2 g, 60% w/w in paraffin oil) was suspended in DMF (400 ml) and the suspension cooled to 5-10° C. Ethyl 2-methyl acetoacetate (Sigma Aldrich; 100 g) was added to the above suspension over a period of 1 hr maintaining the temperature between 5-10° C. The temperature of the reaction mass was then slowly raised to 25-30° C. and the mass is stirred for 1 hr until the evolution of hydrogen had completely ceased. A clear pale yellow color solution with no residual sodium hydride was observed. The reaction flask was then cooled to 10-15° C. and 1,4-butane sultone (Sigma Aldrich; 94.5 g) was added over a period of 1 hr. The temperature of the reaction mass was then slowly raised to 50-60° C. and maintained at 50-60° C. for 15 hrs. After completion of the reaction, the reaction mass was cooled to 5-10° C. and 2-propanol (20 ml) was added to destroy any left over/unreacted sodium hydride. Thereafter the reaction mass was concentrated at 60° C. in vacuo until a thick residue was obtained. The residue was used directly in step (b).

### Step (b)

### 5-Methyl-6-oxoheptane-1-sulfonic acid

The residue from step (a) was dissolved in water (500 ml) and sodium hydroxide (35.6 g) was added. The reaction was heated to 85-90° C. and maintained at 85-90° C. over a period of 15 hrs. Thereafter the reaction mass was cooled to 35° C. and extracted with hexane (2×200 ml) to remove paraffin oil. The pH of the aqueous layer was adjusted slowly to pH 1.0 using 35% aqueous hydrochloric acid. The reaction mass was then concentrated at 60° C. in vacuo until sodium chloride started to precipitate. 2-Propanol (750 ml) was added to the residue and heated to 60° C. Sodium chloride was removed by filtration. The organic layer was concentrated at 60° C. in vacuo. The residue was dissolved in 2-propanol at 60° C. and undissolved sodium chloride was filtered off. The filtrate was concentrated in vacuo at 60° C. until a thick residue was obtained (yield 140 g, 96%).

### Step (c)

### Compound 1

4-Hydrazinobenzene sulfonic acid (Alfa Aesar; 40 g) was suspended in acetic acid (1200 ml) and 5-methyl-6-oxoheptane-1-sulfonic acid [from step (b); 72 g] was added. The reaction mass was heated to 100° C. and maintained at 100° C. for 10-12 hrs during which time the 4-hydrazinobenzenesulfonic acid completely dissolved. Qualitative HPLC analysis of the reaction mass showed less than 1.5% of 4-hydrazinobenzene-sulfonic acid. The reaction mass was cooled to 40° C. and undissolved material removed by filtration. The reaction mass was then concentrated at 60° C. in vacuo until no further acetic acid distilled out. The residue was dissolved in methanol (80 ml) at 55° C. and 2-propanol (800 ml) was added at 55° C. over a period of 30 min. The reaction mass was stirred at 55° C. for a period of 2 hrs. The product was filtered hot under a nitrogen atmosphere and dried under suction. The damp product was dissolved in distilled water (400 ml) at 30° C., and concentrated in vacuo to remove traces of 2-propanol. The pH of the reaction mass was adjusted to pH 8.0 to 8.5 using 10% w/v aqueous sodium hydroxide solution and the reaction mass was heated to 75-80° C. and stirred for 10 hrs. The reaction mass was concentrated at 60° C. in vacuo until no further water distilled out. 2-Propanol (600 ml) was added to the residue and stirred for 30 min. The reaction mass was concentrated in vacuo at 60° C. to dryness, giving Compound 1 as a fine powder. The product was dried for 12 hrs at 60° C. in vacuo. Yield 61.2 g.

### Example 2

### Disodium 2,3-dimethyl-1,3-bis(4-sulfonatobutyl)-3H-indolium-5-sulfonate (Compound 2)

### Step (a)

Compound 1 (fine and dry powder; 53 g) was suspended in N,N-dimethylacetamide (1500 ml) and the suspension heated to 150° C. to achieve dissolution. 1,4-Butane sultone (Sigma Aldrich; 100 g) was added to the solution, and the mixture maintained at 150° C. for 48 hrs. The progress of the reaction was monitored by qualitative HPLC analysis. After every 6 hrs, more 1-4 butane sultone (10 g) was added. After 48 hrs the reaction mass was cooled to 40° C. and the product filtered under nitrogen atmosphere. The damp product was suspended in ethyl acetate (500 ml) and the suspension heated to 60° C. for 1 hr. The reaction mass was then cooled and filtered under a nitrogen atmosphere and dried under suction. The yield of crude product was 55 g.

### Step (b)

### Purification

The crude product from step (a) (55 g) was suspended in methanol (500 ml) and the mixture heated to 65-70° C. (bath temperature). Triethylamine (2 ml) was added and the mixture maintained at 65-70° C. (bath temperature) for 15 min. 0.34% Aqueous isopropanol (1500 ml; 5 ml of water in 1500 ml of 2-propanol) was added to the reaction mass over a period of 30 min during which the product precipitated out. The reaction mass was stirred for 2 hrs. The product was filtered under a nitrogen atmosphere and dried under suction. The purified Compound 2 product was dried in vacuo at 50° C. (yield 45 g).

### Example 3

### Synthesis of 2-[(1E,3E)-4-Anilinobuta-1,3-dienyl]-3-methyl-1,3-bis(4-sulfobutyl)-3H-indolium-5-sulfonate (Compound 3)

Compound 4 (20 g) and Malonaldehyde bis(phenylimine).HCl (Compound Z; Sigma Aldrich; 10.8 g) were suspended in a mixture of acetic anhydride (100 ml) and acetic acid (300 ml) and the contents heated to 110° C., and maintained at 110° C. for a period of 15 hrs. Thereafter, the reaction mass was cooled to 60° C. and distilled under vacuum. The residue was dissolved in acetic acid (100 ml) poured into ethyl acetate (1000 ml) and stirred for 2 hrs at room temperature (25-30° C.). The precipitated solid was filtered under a nitrogen atmosphere and dried under suction. The crude material was then suspended in methanol (100 ml) and heated to reflux. 2-Propanol (500 ml) was added slowly to the reaction mass and stirred for 1 hr. The product was filtered under a nitrogen atmosphere and dried under suction. The Compound 3 was dried in vacuo at 40° C. Yield 22 g (88%).

### Example 4

### Preparation of 1-(ε-Carboxypentyl)-2,3,3-trimethyl indolenium-5-sulfonate (Compound 4)

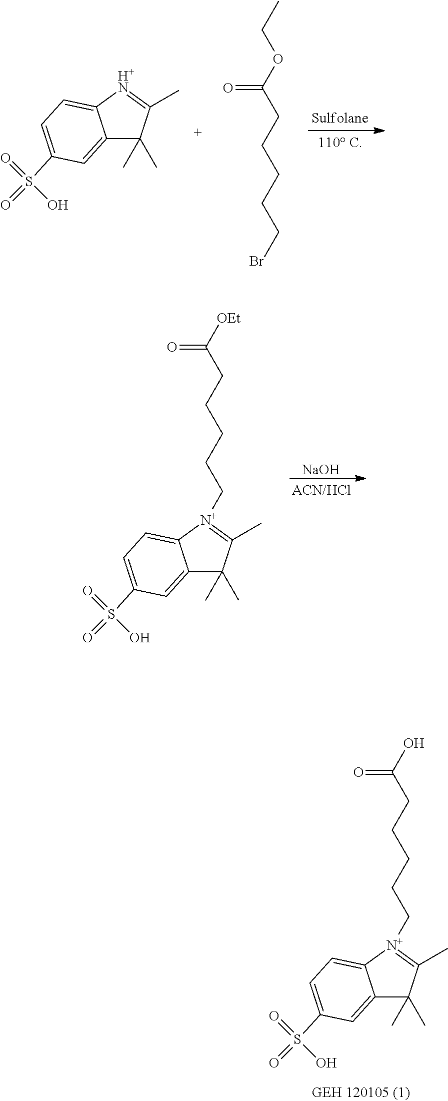

Sulfo-2,3,3-trimethyl indolenine sodium salt (purchased from Inta. Trade; Compound 1; 100 g, 0.38 μmol) was charged into a reaction vessel (3 L). Sulfolane (Sigma-Aldrich; 400 ml) was added to it. Ethyl-6-bromo hexonate (Sigma-Aldrich; 140 ml, 0.760 mol) was then added to the mixture. The contents were heated to 110° C. with stirring and the reaction mixture was maintained at this temperature for 16 hrs. The reaction mixture was then cooled to ˜25° C. Ethyl acetate (1 L) was added. The mixture was stirred for 15 min, and the ethyl acetate layer was decanted. The reddish brown residue in the flask was washed with ethyl acetate (300 ml×2). Deionised water (1 L) was added and the mixture was stirred for 15 min to obtain a clear solution.

The solution was washed again using fresh ethyl acetate (500 ml×2). Traces of ethyl acetate were removed from the resulting solution on the rotary evaporator. Sodium hydroxide (36 g, 0.90 mol) was added to the above solution and pH maintained between 10-12 (Note: pH>10 is critical for the completion of reaction). The reaction mixture was heated to 70° C. for 1 hr. The reaction mixture was neutralized using HCl (60 ml) and evaporated water to dryness on the rotary evaporator. The material was dried in vacuo at 30° C. for 16 hr. Acetonitrile (900 ml) was added to the above dried material. Concentrated hydrochloric acid (126 ml) was added slowly with stirring. The mixture was stirred at ˜25° C. for 15 min. The suspension was filtered, washed with 9:1 acetonitrile/conc. HCl (100 ml×2) and dried.

The filtrate was concentrated to a viscous mass (Note: completely dried material takes longer time to crystallize and risks formation of the isopropyl carboxylate ester of Compound 4). The residue obtained was triturated with a mixture of isopropyl alcohol/acetone (150 ml/350 ml) for 15 min (the mixture is to be filtered immediately, to avoid the formation of the isopropyl ester). The suspension was filtered, washed with a mixture of isopropyl alcohol/acetone mixture (30/70 ml) and dried under vacuum at 50° C. for 8 hrs. Yield: 90 g (66%). HPLC Purity: 95% (at 270 nm).

### Example 5

### Preparation of 3,3-Dimethyl-5-sulfo-1,3-dihydro-indol-(2E)-ylidenel-penta-1,3-dienyl}-3-methyl-5-sulfo-1,3-bis-(4-sulfobutyl)-3H-indolium (Compound 5; Cy5**)

Compound 3 (22 g) and Compound 4 (12.5 g) were suspended in a mixture of acetic anhydride (130 ml) and acetic acid (370 ml), and the contents heated to 110° C. Potassium acetate (6.5 g) was added, and the reaction mass maintained at 110° C. for 4 hrs during which all of Compound 3 was consumed. The reaction mass was cooled and concentrated at 60° C. in vacuo. The residue was dissolved in acetic acid (100 ml) and poured into ethyl acetate (750 ml) and stirred for 2 hrs at room temperature. The precipitated solid was filtered under a nitrogen atmosphere and dried under suction. The product was dissolved in water (100 ml) and stirred for 2 hrs monitoring the hydrolysis of anhydride (by HPLC). Any traces of ethyl acetate were removed either in vacuo, or by separation, and the aqueous layer was freeze-dried. Dry wt of the product=29.8 g (yield ca. 100%).

### Example 6

### HPLC Purification and Analysis of a Dye Composition of the Invention

### Preparative Separations:

### Sample Preparation:

Two bottles, each containing 2-5 g of Compound 5, were dissolved in 0.1% TFA (50 ml) in water. The pH was adjusted to 2.1 with ˜1M THF in water (0.8 ml TFA in 10 ml water) and filtered before purification on the LaPrep system. 4 runs were carried out.

### System Parameters:

Runs were performed as described in table below.

| System | LaPrep |
|---|---|
| Sample | AH114444, FEK319/053, JHJ. |
| Column | Luna C18 10 μm, 250 × 75 mm |
| Mobile phase A | 0.1% TFA in H₂O |
| Mobile phase B | 0.1% TFA in acetonitrile 20 |
| Gradient | 2% B in 7 minutes, 2-35% B in 28 min. |
| Flow [ml/min] | 175 |
| Pressure [bar] | 71, 76, 72, 71. |
| Detection UV [nm] | 254 |
| Injections | 1-2.5g × 4 |

### Analytical Method:

### Column: XBridge Shield RP18, (4.6×50), 2.5 μm, Waters

Mobile phase-A: 10 mM ammonium acetate, pH ˜6.8
Mobile phase-B: 10% MF-A and 90% acetonitrile

### Gradient:

| | Time (min) | | | | | | |
|---|---|---|---|---|---|---|---|
| | 0 | 2 | 6 | 7 | 9 | 9.1 | 12 |
| % B | 3 | 3 | 25 | 100 | 100 | 3 | 3 |

Flow: 1.0 ml/min.
Column oven: 40° C.
Sample concentration: 0.020 mg/ml in water.

What is claimed is:

1-14. (canceled)

15. A composition which comprises an unsymmetrical cyanine dye of Formula A, together with symmetrical cyanine dyes of Formula B and Formula C: 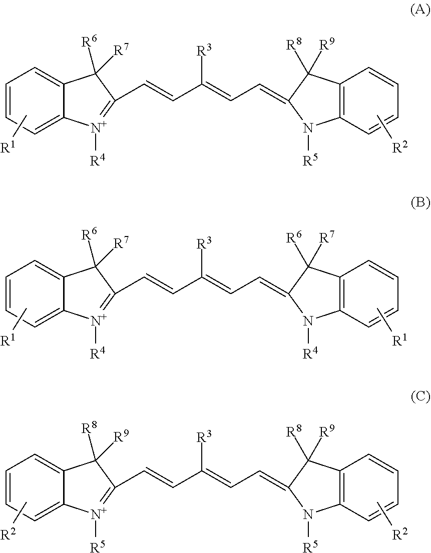
wherein:c
the molar ratio of A:B and also A:C in said composition is at least 46:1;
R¹ and R² are independently H, —SO₃M¹ or R^{a}, where M¹ is H or B^{c}, and B^{c} is a biocompatible cation;
R³ is H or an R^{d} group;
R⁴ and R⁵ are independently an R^{d} group;
R⁶ to R⁹ are independently C₁₋₃ alkyl or an R^{c} group, and are chosen such that in Formula A, at least one of R⁶ to R⁹ is R^{c};
R^{a} is C₁₋₄ sulfoalkyl;
R^{b} is C₁₋₆ carboxyalkyl;
R^{c} is an R^{a} or R^{b} group;
R^{d} is C₁₋₅ alkyl or an R^{c} group;
with the provisos that in Formula A:
(i) at least one of the pairs of substituents R¹/R², R⁴/R⁵, R⁶/R⁸, and R⁷/R⁹ is different;
(ii) the cyanine dye comprises at least one R^{b} group and a total of 3 to 6 sulfonic acid substituents from the R¹, R², R^{a} and R^{c} groups.

16. The composition of claim 15, where R³ is H.

17. The composition of claim 15, where R⁶ to R⁹ are independently CH₃ or an R^{c} group.

18. The composition of claim 15, which further comprises an acylated indole of Formula H: 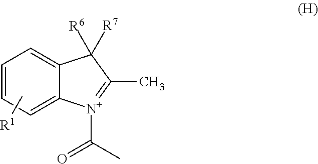
wherein:
R⁶ and R⁷ are independently C₁₋₃ alkyl or an R^{c} group, and are chosen such that in Formula H, at least one of R⁶ and R⁷ is an R^{c} group;
R¹ and R^{c} are as defined in claim 15; and
the molar ratio of A:H is at least 92:1.

19. The composition of claim 15, which further comprises sulfonate ester derivatives of Formula A¹, B¹ or C¹, which correspond to the dyes of Formula A, B or C respectively wherein at least one of R¹ and R² is —SO₂—OR^{d}, where R^{d} is C₁₋₅ alkyl or an R^{c} group; R^{c} is an R^{a} or R^{b} group; R^{a} is C₁₋₄ sulfoalkyl; and R^{b} is C₁₋₆ carboxyalkyl; such that the molar ratio of A: (A¹+B¹+C¹) is at least 92:1.

20. A composition which comprises a hemicyanine dye of Formula E, and an amidine salt of Formula Z: 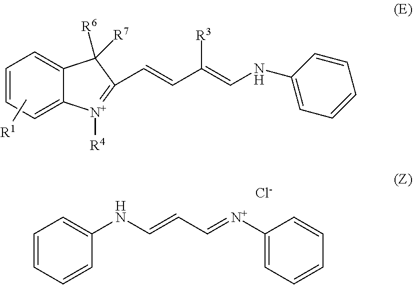
wherein:
R¹, R³, R⁴, R⁶ and R⁷ are as defined in claim 15; and
the molar ration of E:Z in said composition is at least 46:1.

21. A composition which comprises an indolinium salt of Formula F, and an indole of Formula G: 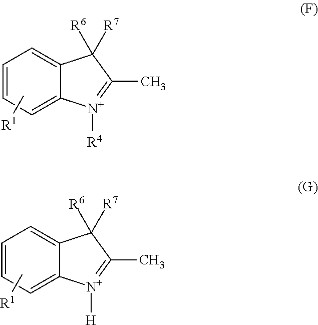
wherein:
R¹ and R⁴ are as defined in claim 15;
R⁶ and R⁷ are independently C₁₋₃ alkyl or an R^{c} group, and are chosen such that in Formulae F and G, at least one of R⁶ and R⁷ is an R^{c} group; and
the molar ratio of F:G in said composition is at least 92:3.

22. A method comprising reaction of the composition of claim 21 with 1.05 to 1.25 molar equivalents of the amidine salt of Formula Z:

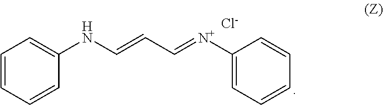 [SMILES: [Cl-] ; C(=C\Nc1ccccc1)/C=[NH+]/c1ccccc1]

23. A composition comprising:
an indole of Formula G: 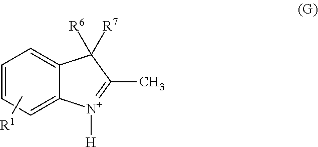
wherein:
R¹ and R⁴ are as defined in claim 15;
R⁶ and R⁷ are independently C₁₋₃ alkyl or an R^{c} group; and
an acylated indole of Formula H: 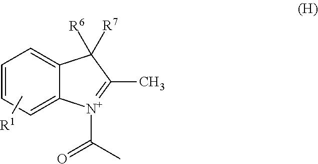
wherein:
R¹ is as defined in claim 15;
R⁶ and R⁷ are independently are independently C₁₋₃ alkyl or an R^{c} group, and are chosen such that in Formulae G and H, at least one of R⁶ and R⁷ is an R^{c} group; and
the molar ratio of G:H in said indole composition is at least 18:1.

24. A method comprising reaction of the composition of claim 23 with an alkylating agent of formula R⁴—X, where R⁴ is an R^{d} group; R^{d} is C₁₋₅ alkyl or an R^{c} group; R^{c} is an R^{a} or R^{b} group; R^{a} is C₁₋₄ sulfoalkyl; R^{b} is C₁₋₆ carboxyalkyl; and X is a leaving group.

25. A method comprising reaction of a composition which comprises an indolinium salt of Formula F, and an indole of Formula G: 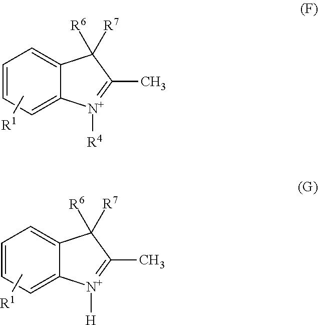
wherein:
R¹ and R⁴ are as defined in claim 15;
R⁶ and R⁷ are independently C₁₋₃ alkyl or an R^{c} group, and are chosen such that in Formulae F and G, at least one of R⁶ and R⁷ is an R^{c} group; and
the molar ratio of F:G in said dye head group composition is at least 92:3;
with one molar equivalent of an indolinium salt of Formula J: 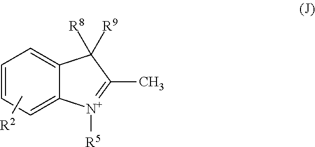
wherein:
R² and R⁵ are as defined in claim 15;
R⁸ and R⁹ are independently C₁₋₃ alkyl or an R^{c} group.

26. A pharmaceutical composition which comprises the composition of claim 15, in a biocompatible carrier medium, in sterile form suitable for mammalian administration.
